(19) United States
(12) Patent Application Publication
(10) Pub. No.: US 2010/0203649 A1
(43) Pub. Date: Aug. 12, 2010

(54) OPTOCHEMICAL SENSOR ELEMENT

(75) Inventor: Rolf Thrier, Tagelwangen (CH)

Correspondence Address: STANDLEY LAW GROUP LLP, 6300 Riverside Drive, Dublin, OH, 43017, US

(73) Assignee: Mettler-Toledo AG, Greifensee (CH)

(21) Appl. No.: 12/698,362

(22) Filed: Feb. 2, 2010

Related U.S. Application Data
(63) Continuation of application No. PCT/EP2008/060075, filed on Jul. 31, 2008.

(30) Foreign Application Priority Data
Aug. 2, 2007 (EP) 07113709.5

(51) Int. Cl. G01N 33/00; G01N 31/22; G01N 21/64

(52) U.S. Cl. 436/127; 422/82.07; 422/86

(57) ABSTRACT

An optochemical sensor element 9 measures gaseous or dissolved analytes, in particular of oxygen. The sensor element 9 includes a fluorophor 25 that is immobilized in a polymer matrix 23. The polymer matrix itself is formed of a polymer with a non-aromatic backbone. The sensor element 9 is useful in a measuring device.

### CROSS-REFERENCE TO RELATED APPLICATIONS

This application is a continuation under 35 USC §120 of PCT/EP2008/060075, filed 31 Jul. 2008, which is in turn entitled to benefit of a right of priority under 35 USC §119 from European patent application 07 11 3709.5, which was filed 2 Aug. 2007. The content of each of the applications is incorporated by reference as if fully recited herein.

### TECHNICAL FIELD

The disclosed embodiments relate to optochemical sensor elements for the measurement of gaseous or dissolved analytes, in particular of oxygen, and to methods involving the use of such sensors.

### BACKGROUND OF THE ART

The need to determine the concentration of gaseous or dissolved analytes occurs in a multitude of applications and processes. For example, the monitoring of the oxygen concentration in biotechnological processes is indispensable for the control of the processes. This also applies to a number of further analytes, such as for example CO₂, SO₂, H₂O₂ or nitrogen oxide.

Several of the known methods for the measurement of such analytes have the disadvantage that they cause a change in the concentration of the analyte during the measurement, as the analyte is being consumed in the measurement. As an alternative to measurement methods of this kind, there have been recent developments of optochemical measurement methods. The latter methods are based on a sensor element whose properties change dependent on the quantity of analyte that is present. One such property that is used in optochemical sensor elements of this type is the fluorescence which can be for example excited or extinguished—i.e. quenched—by the analyte.

An optochemical sensor element, described in U.S. Pat. No. 6,432,363 B2, contains a luminescent dye that is immobilized in a polymer matrix. The polymer matrix is free of plasticizers and includes at least one polymer with phenyl groups in the main chain.

An optical sensor element for the determination of ions in aqueous solutions is disclosed in WO 95/26501 A1. The sensor element contains a transparent substrate which is coated with a hydrophobic polymer with a glass transition temperature (Tg) in the range from −150° C. to 50° C. The fluorophor is in this case immobilized in the coating.

In U.S. Pat. No. 5,387,525 A, a method is described for activating the fluorescence of poly-anionic fluorophors through quartary ammonium salts such as tetra-butyl ammonium hydroxide as well as the use of the method in an optochemical sensor element.

In WO 2004/027412 A1, a sensor element is described which discolors under the influence of an analyte and thus can be used for example as an indicator for the spoiling of food products. The sensor element includes a transition metal complex, which is immobilized in a matrix and which can also be a fluorescent dye or a fluorophor.

Y. Amao et al. (Analytica Chimica Acta 407 (2000), 41-44) describe a fluorescence-based sensor element for oxygen, in which an Al-phthalocyanin fluorophor is used which is immobilized in a polystyrene film.

Y. Amao et al. (Analytica Chimica Acta 421 (2000), 167-174) describe the measurement of oxygen content by using the luminescence changes of metalloporphyrins that are immobilized in a film of poly(isobutyl methacrylate-co-trifluoro methacrylate).

Y. Amao et al. (Analytica Chimica Acta 445 (2001), 177-182) describe a luminescent iridium(III) complex, immobilized in a polymer film, as a material for the optical determination of oxygen content.

In a publication by M. Florescu, A. Katerkamp (Sensors and Actuators B 97 (2004), 39-44), the optimization of polymer membranes for the optical measurement of oxygen content is described. For the fluorophor a ruthenium-metal complex, Ru(dpp)₃Cl₂, was used.

A disadvantage that has been found in the optochemical sensor elements described so far is their rather limited stability when they are subjected to cleaning processes and to sterilization in the process system, for example to autoclaving procedures, CIP (cleaning-in-place) treatments, SIP (sterilizing-in-place) treatments, and also their inadequate stability when they are exposed to process media that contain for example polar organic solvents. Particularly for sensor elements that are used in biotechnology, the cleaning, autoclaving and CIP/SIP treatments of the measurement apparatus equipped with the sensor elements are of importance. The high temperatures involved in sterilizing, specifically autoclaving, often cause a loss of immobilized fluorophors, for example due to thermal disintegration and bleaching of the fluorophor as well as due to washing-out of the fluorophor from the carrier matrix. Especially in polymers with a low glass transition temperature, the high temperatures lead to an increased mobility of the polymer chains in the polymer matrix and thus to an increased diffusion of the fluorophor through the polymer matrix, and consequently to an increased wash-out. Furthermore, especially the polymer matrices named in U.S. Pat. No. 6,432,363 B2, due to the aromatic character of the polymer backbone, have an inherent color which affects their optical transmissivity or transparency and thus makes the fluorescence measurement more difficult. Such an inherent coloring can also occur as a result of aging processes which are caused by the influence of temperature or humidity and which lead for example to a yellowing of the polymer matrix.

### SUMMARY

It is an objective to provide an improved optochemical sensor element which has a high mechanical stability and is very stable in withstanding cleaning processes and process media and which, consequently, is in essence resistant to aging and has a long service life. The sensor element should also have essentially no inherent coloring caused by the polymer matrix. An additional aim is to provide an economical alternative to the commercially available optochemical sensor elements, which preferably also meets the requirements for use in critical biological and biochemical processes and is compatible with the substances used in these fields.

The concept of stability encompasses different aspects. Preferably, the optochemical sensor element is thermally stable, and the fluorophor does not bleach out or wash out under thermal exposure nor due to aging caused by its use in the process, by CIP procedures, or autoclaving. Furthermore, the polymer matrix should not change its optical and mechanical properties over time and it should not become yellow, brittle, or exhibit similar effects of aging. In order to keep its ability to function, the polymer, specifically the polymer matrix, is preferably resistant to interfering substances such as water, ions and/or solvents even after an aging process has taken place, so that damage to the polymer matrix and to the fluorophor imbedded in it can be prevented. In addition the polymer matrix should have the stability to withstand the solvents that are being used without swelling up, it should have a constant concentration and uniform distribution of the fluorophor and also exhibit a constant Stern-Vollmer characteristic.

This and other objects are solved by an improved optochemical sensor element in accordance with the independent claim. Further preferred embodiments are the subjects of the dependent claims.

An optochemical sensor element includes a suitable fluorophor which is immobilized in a polymer matrix. The polymer that is used to form the polymer matrix comprises a non-aromatic backbone. The layer thickness of the polymer matrix is preferably in the range of about 3 to 10 μm.

The term “backbone” describes the main chain of the polymer. In other words, only the main chain of the polymer is non-aromatic. Side chains or side groups can also comprise aromatic components.

The term “analyte” describes the substance that is to be measured, specifically oxygen.

Due to the non-aromatic backbone, the inherent coloring of the polymer matrix can be reduced to a low level, and the best possible optical transparency of the polymer matrix can be achieved. The following polymers can be used to form the polymer matrix of optochemical sensor elements: cyclic olefin copolymers (“COC”) such as ethylene-norbornene copolymer, cyclic olefin polymers (“COP”) and poly(n-methyl methacrylimide) (PMMI). It is also possible to use mixtures of these polymers.

The use of these polymers as polymer matrix in sensor elements is advantageous, as they have particularly high mechanical stability and are very stable in withstanding acidic and basic cleaning processes, so that as a result the sensor elements have a long service life.

The choice of the fluorophor depends on the analyte that is to be measured and its solubility in the polymer matrix. Suitable fluorophors are those with a long fluorescence lifetime and those whose fluorescence exhibits a strong dependency on the concentration of the analyte that is to be measured. Examples of suitable fluorophors, in particular for the measurement of oxygen content, include Pt(II)-meso-tetra(pentafluorophenyl)-porphine, Pt(II)-5,10,15,20-tetrakis-(2,3,4,5,6-pentafluorophenyl)-porphyrin (PtTFPP), Pt(II)-octaethyl porphyrin (PtOEP), Pt(II)-octaethyl porphyrin ketone (PtOEPK) and, analogous to these compounds, the Pd(II)-complexes PdTFPP, PdOEP and PdOEPK, as well as Pd(II)-meso-tetraphenyl-(tetrabenzo)porphine (PdTPTBP). Other fluorophors that can be used include Ir(III)((N-methyl-benzoimidazol-2-yl)-7-(diethylamino)-cumarin))₂(acac), Ir(III)((benzothiazol-2-yl)-7-(diethylamino)-cumarin))₂-(acac). Numerous further suitable fluorophors are commercially available.

It is further advantageous if the fluorophors that are used have an adequate solubility so that they can be dissolved with sufficient concentration in the solvents used to produce the polymer matrix and also in the polymer as well as in the polymer matrix itself. Preferably, the dissolved fluorophors are present in these solvents as well as in the polymer matrix in an essentially non-agglomerated state and are with preference homogeneously dissolved.

Consequently, the aforementioned hydrophobic porphyrin complexes are particularly well suited for use as fluorophors in a hydrophobic COC or COP matrix. In PMMI matrices it is also possible to use fluorophors that are more hydrophilic.

Further usable as fluorophors are different ruthenium- or osmium complexes such as tris(phenanthroline)Ru(II)-chloride, tris(4,7-diphenyl-1,10-phenanthroline)Ru(II)-TMS or tris(4,4-diphenyl-2,2-bipyridine)Ru(II)-chloride. These hydrophilic fluorophors can likewise be used in the polymer matrices named herein. However, in order to reduce their hydrophilic property, they should be used together with a lipophilic counter-ion, for example tetraphenyl borate.

The optochemical sensor elements exhibit outstanding stability against cleaning and sterilizing processes, for example autoclaving (30 minutes at 130° C. in water vapor atmosphere), or CIP treatments (30 to 60 minutes with 3% NaOH solution at 90° C.).

In a preferred embodiment, the polymer is selected from ethylene-norbornene copolymers and poly(n-methyl methacryl-imide). These polymer matrices are commercially available from TOPAS Advanced Polymers GmbH of Germany under the registered trademark TOPAS® (ethylene-norbornene copolymers) and PLEXIMID™ (poly(n-methylmethacrylimide)), which is commercially available from Evonik Degussa.

As will be described in the following paragraphs, these polymers have a number of properties that make them well suited as polymer matrices to immobilize fluorophors for autoclavable optochemical sensor elements, in particular oxygen-sensitive sensor elements.

The polymers have very good optical transparency, allowing optical radiation to pass through the polymer matrix to excite the fluorophor. The optical transparency (according to ISO 13468-2) for TOPAS is around 91% (types: 5013X25 with a glass transition temperature Tg=134° C.; 6013S-04 with Tg=138° C.; 6015S-04 with Tg=158° C.; 6017S-04 with Tg=178° C.; 5013S-04 with Tg=134° C.; TKX-0001 with Tg=134° C.) and for PMMI around 90-91%. Alternatively, the optical grades of TOPAS are characterized by their haze values according to ISO 14782. The TOPAS quality grades 5013X14 (Tg=136° C.) and 6013F-04 (Tg=140° C.) have haze values <1%.

The polymers have good stability under light- and/or radiation exposure, so that the optical transparency of the polymer matrix remains unaffected even after exposure of the sensor element to radiation suitable for the excitation of the fluorophor. Suitable fluorophors are excited for example with radiation in the near UV range and/or UV-VIS range, preferably with a wavelength between about 320 nm and about 700 nm.

The polymers also exhibit a very high stability against gamma radiation and gamma sterilization, and also against sterilization with ethylene oxide. These are frequently used methods for the sterilization of sensor elements. The optochemical sensor elements, in particular the polymers used in them, have to withstand these treatments without yellowing nor showing other degradations such as for example becoming brittle, hydrophilizing, or a decay of the polymer, a chain length degradation, or an uncontrolled cross-linking of the polymer.

In addition, polymer matrices of ethylene-norbornene copolymers or poly(n-methylmethacrylimide) are also stable against steam sterilization procedures (130° C. in H₂O steam atmosphere).

The preferred polymer matrices have a sufficiently high oxygen permeation (250 cm³×100 μm/(m²×day×bar) for TOPAS 5013X14 (Tg=136° C.) and 280 cm³×100 μm/(m²×day×bar) for TOPAS 6013F-04 at 23° C. and 50% relative humidity) (according to ASTM D3985). Thereby, optochemical sensor elements with short response times can be realized.

The preferred polymer matrices further exhibit only a limited degree of water absorption, water vapor permeability and a limited tendency to swell in polar solvents such as acetone or isopropanol. This is advantageous as water and different solvents can change the matrix and its Stern-Vollmer characteristic. Furthermore, with increasing water absorption one also has to expect a diffusion of ions, in particular foreign ions, into the polymer matrix which would cause interference and therefore strongly needs to be avoided. An absorption of water or solvent further leads to an undesirable swelling of the polymer matrix which in final consequence affects the fluorophor concentration in the polymer or, more specifically, in the polymer matrix and can lead to measurement inaccuracies. The polymer matrix thus also functions as a membrane insofar at it is permeable on the one hand for the analyte, for example oxygen, but holds off on the other hand the passage of water, H₂O steam, solvents and ions, so that a degradation of the fluorophor by penetrating substances can be avoided.

For TOPAS® (ethylene-norbornene copolymers) the following values are published for water absorption and water vapor permeation, respectively:

Water absorption (according to ISO 62):

0.01% for all types of TOPAS

water vapor permeation (38° C., 90% relative humidity):

| | |
|---|---|
| 1.0 g × 100 μm/(m²×day) | (TOPAS type 5013X14) |
| 1.3 g × 100 μm/(m²×day) | (TOPAS type 6013F-04) |

Conventional polymer matrices that are used to immobilize fluorophors have significantly higher values for water absorption, for example 0.25% for polyetherimides (PEI), 0.24% for PSU, and 0.4% for polyethersulfone.

Furthermore, the fluorophors have good solubility in the preferred polymer matrices. The concentration of the fluorophor that is dissolved in the polymer is sufficiently high to achieve a good fluorescent response for thin sensor elements and a fast, i.e. short, response time.

Further suitable selections of polymers are cyclic olefin polymers (COP), which are commercially available from Zeon Corporation of Japan under the trade names of ZEONEX® or ZEONOR®, or poly(n-methyl methacrylimide), which is commercially available under the trade name of PLEXIMID®.

The following properties are published for these polymers:

| | ZEONEX ® | ZEONOR ® | PLEXIMID ® |
|---|---|---|---|
| optical transparency [%] | 92 | 92 (with 3 mm | 90 |
| | | thickness) | |
| water absorption [%] | <0.01 | <0.01 | 0.4 |
| glass transition | 70-163 | 70-163 | 172 |
| temperature [° C.] | | | |
| oxygen permeability | | ca. 1 × 10⁻³ | |
| [cm³ × cm/(cm² × | | | |
| s × cmHg)] | | | |
| water vapor permeation | 1-2.5 | ca. 1 | |
| 40° C., 90% RH | | | |
| [g × 100 μm/(m² × day)] | | | |

The glass transition temperature of ZEONEX® and ZEONOR® can be adjusted depending on the actual composition of the polymers.

To allow the film formation out of a solution, it is advantageous if the polymer is soluble in an appropriate solvent with a high vapor pressure. It is important that the selected fluorophor likewise dissolves in sufficient concentration in the same solvent in order to achieve a uniform distribution of the fluorophor in the polymer matrix. Possible solvents for the polymers are chloroform and cyclohexane.

In a preferred embodiment of the optochemical sensor element, the polymer matrix comprises ethylene-norbornene copolymers.

The ethylene-norbornene copolymers (cyclic olefin copolymers, COC) have high glass transition temperatures (Tg). With glass transition temperatures above 130°, which is the temperature normally chosen for autoclaving, one prevents the problem that the polymer chains could become mobile during an autoclaving cycle and the fluorophor could diffuse out of the matrix. A loss of fluorophor is thereby practically avoided, which results in a long service life of the sensor element. By varying the proportion of norbornene in the copolymer, one can furthermore influence the glass transition temperature of the polymer and thus adapt it for the desired purpose.

In further preferred embodiments of the optochemical sensor element, the polymer matrix comprises cyclic olefin polymers (ZEONEX®, ZEONOR®; COP) or poly(n-methyl methacrylimide) (PMMI).

In a further preferred embodiment, the polymer matrix is applied on a substrate. As a result, the optochemical sensor element is very resistant to mechanical stress, for example elevated pressure levels or pressure fluctuations in bioreactors.

Preferred materials from which substrates can be made are glass, polyester, amorphous or partially crystalline polyamides, polyacrylates, polycarbonates, COC-polymers (TOPAS), COP-polymers (ZEONOR, ZEONEX), and poly(n-methyl methacrylimide). COC-polymers are available under the trade name of TOPAS (Ticona Polymer), COP-polymers under the trade names of ZEONOR or ZEONEX (Zeon Chemicals), and poly(n-methyl methacrylimide) under the trade name of PLEXIMID (Röhm GmbH).

Besides the carrier materials that have already been mentioned above, it is also possible to use hybrid substrates comprising of combinations of these materials, such as for example a glass-polymer compound material.

The ethylene-norbornene copolymers which belong to the cyclic olefin copolymers (COC) are preferred as materials for the substrate, as they show essentially no inherent fluorescence and very good optical transparency which also still remains preserved after an aging process.

The sensor element in a further preferred embodiment has a cover layer. This cover layer has to be permeable to the analyte but impermeable to extraneous radiation, so that an undesirable influence on the fluorescence measurement from extraneous light or from fluorescence of the measurement sample can be avoided. Possible materials for the cover layers are white, porous TEFLON® coatings (thickness of 5 μm) or white oxygen-permeable silicone coatings or white porous papers.

In a further embodiment, the polymer matrix is imbedded in a silicone film in the form of polymer spheres or matrix fragments. The fluorophor is immobilized directly in the polymer spheres or matrix fragments. This arrangement is advantageous as the spheres or fragments imbedded in the silicone will enhance the fluorescence, due to diffuse light-scattering.

The analyte in a preferred embodiment is gaseous or dissolved oxygen, which is for example dissolved in aqueous solutions or media, or dissolved in media containing low proportions of polar solvents such as for example methanol, ethanol, acetone or isopropanol.

In further embodiments, the analyte is gaseous or dissolved O₂, gaseous or dissolved H₂O₂, SO₂ or nitrogen oxide.

Particularly for the determination of gaseous or dissolved oxygen, it is advantageous if the substrate is impermeable to oxygen and its ability to dissolve oxygen is as low as possible. This ensures that the optochemical sensor element will function properly and can be operated essentially without drift and with a short response time. The substrate preferably has a significantly lower oxygen permeability and oxygen solubility than the polymer matrix in which the fluorophor is immobilized.

Sensor elements can be manufactured with different methods, as will be described in the following in context with several examples.

In the process of manufacturing the sensor elements, the layer thickness of the polymer matrix with the immobilized fluorophor affects two important quantities in the optical measurement of analytes by means of fluorescence quenching. On the one hand, the layer thickness determines the emission rate or the reflection rate and thus the intensity of the fluorescent response, and on the other hand it is a determining factor for the response time of the measuring device.

In a preferred embodiment the polymer matrix with the fluorophor is formed as a film either directly on a substrate or applied to the latter subsequent to the film formation. In order to achieve a good bonding between the film and the substrate, adhesion agents or adhesives are used with preference. The polymer matrix can be applied to a substrate by different methods such as dip-coating, spin-coating, or blade coating.

It is also possible to apply a polymer matrix with immobilized fluorophor directly on an optical fiber core. This concept is of interest especially for brittle polymer types such as for example TOPAS (Tg=180° C.) which should as much as possible be kept stress-free and thus free from damage for example by chipping after having been applied to the substrate carrier. The application on an optical fiber core is possible for example with TOPAS. To accomplish this, the polymer matrix with the immobilized fluorophor is arranged directly on the core of a Polymer Optical Fiber (“POF”) optical fiber whose core preferably comprises a polymer with a high refractive index, such as for example poly(pentabromophenyl acrylate-co-glycidyl methacrylate).

### BRIEF DESCRIPTION OF THE DRAWINGS

Schematic illustrations of the embodiments are attached, wherein identical parts are identified by identical reference numbers and wherein:

FIG. 1 shows an optochemical measuring device with a sensor element;

FIG. 2 shows enlarged detail of the optochemical sensor element of FIG. 1;

FIG. 3 shows a further embodiment of the optochemical sensor element; and

FIG. 4 shows a further embodiment of the optochemical sensor element.

### DETAILED DESCRIPTION OF THE PREFERRED EMBODIMENTS

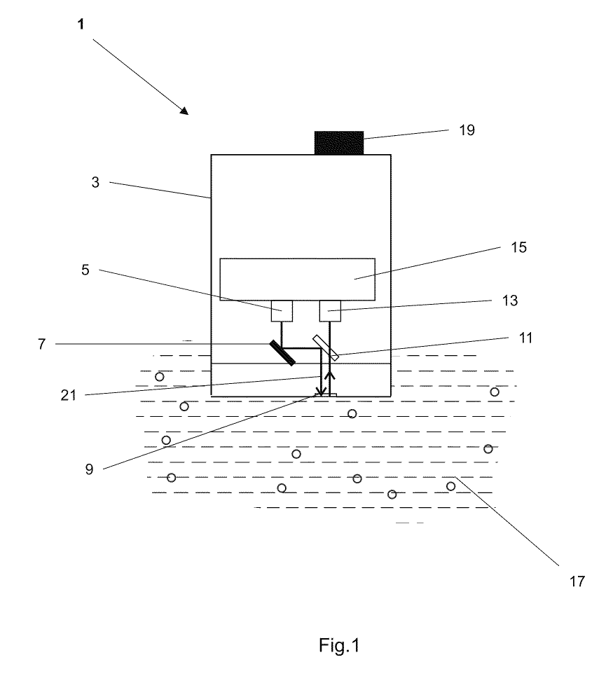

FIG. 1 shows the principal arrangement of an optochemical measuring device 1 with a sensor element 9. The measuring device 1 has a housing 3 containing a radiation source 5, for example a light emitting diode (“LED”), a mirror 7, the optochemical sensor element 9, a beam splitter or filter 11, a detector 13 and an electronic measuring circuit 15. The radiation emitted by the radiation source 5, the excitation light or the excitation radiation, is directed by way of the mirror 7 and the beam splitter 11 to the optochemical sensor element 9. The optochemical sensor element 9 is in contact with the medium 17 which contains the analyte to be measured. The fluorescence emitted after excitation of the fluorophor in the optochemical sensor element 9 passes through the beam splitter 11 without being deflected and falls on the detector 13, whose signal is processed by the electronic measuring circuit 15 and passed on by way of a transmitter or an interface 19. The optical path 21 is indicated only in a schematic fashion.

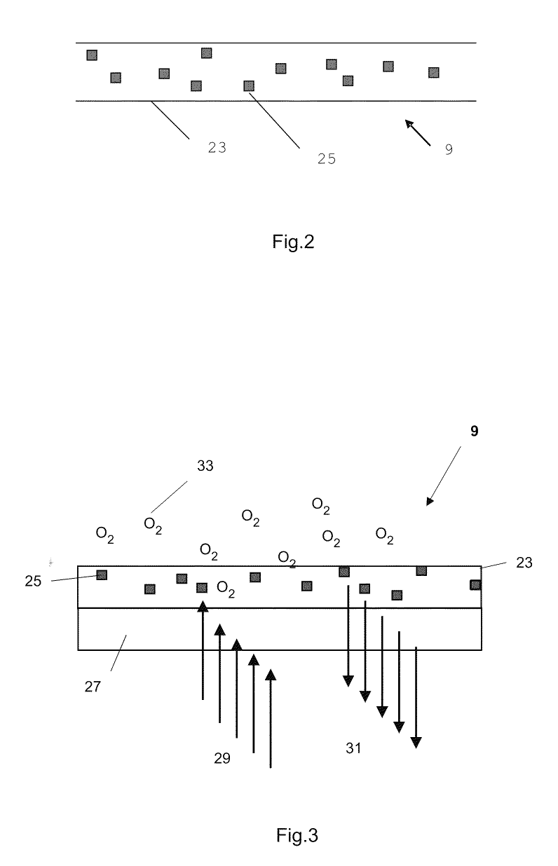

As an enlarged detail of the optochemical measuring device 1, FIG. 2 represents the optochemical sensor element 9 in its simplest embodiment. It comprises a polymer matrix 23 and, immobilized in the latter, a fluorophor 25.

FIG. 3 illustrates an embodiment of the optochemical sensor element 9 of the optochemical measuring device 1. The polymer matrix 23 in which the fluorophor 25 is immobilized is arranged on a substrate 27. Said substrate 27 provides the necessary mechanical stability to the membrane 9 to withstand for example pressure fluctuations. Also shown in this drawing is the excitation radiation 29 which, after passing through the substrate 27, falls onto the fluorophor 25 that is immobilized in the polymer matrix 23, and the fluorescence 31 emitted by the fluorophor. The intensity of the fluorescent response is influenced by the analyte 33, for example oxygen.

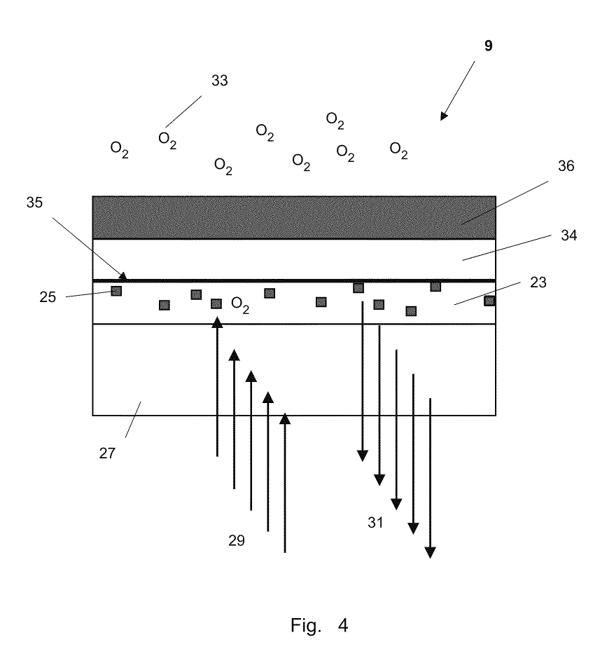

In FIG. 4, a further embodiment of the optochemical sensor element 9 is shown which comprises at least one additional cover layer (two layers 34, 36 in the illustrated example) to separate the polymer matrix 23 from the medium. This cover layer 34, 36 is preferably permeable to the analyte and impermeable to extraneous radiation, so that extraneous radiation arriving from the outside is preferably reflected at the cover layer 34, 36 and does not penetrate into the polymer matrix. 23.

The sensor element 9 comprises a glass wafer as substrate 27 on which, as already shown in FIG. 3, a polymer matrix 23 is arranged in which a fluorophor 25 is immobilized. The polymer matrix 23 is in essence optically transparent. The polymer matrix 23 is adjoined by a first cover layer 34 which in this case is for example a white silicone layer. This first cover layer 34 essentially reflects the excitation radiation 29 so that a high fluorescence yield and short response times can be realized. Preferably, the emitted fluorescence or fluorescent response 31 is reflected almost completely at the boundary layer 35 between the first cover layer 34 and the polymer matrix 23 or diffusely scattered and then directed to the detector (see FIG. 1).

In addition, the sensor element 9 can be covered, as shown here, with a further cover layer 36 which is for example configured as a black silicone layer. The further cover layer 36 is preferably permeable for the analyte and impermeable to radiation, so that fluorescent radiation cannot escape from the sensor element into the measuring medium, and the measurement performed with the sensor element 9 cannot be disturbed by extraneous radiation entering from the outside. The term “white silicone” as used here means silicone with an addition of for example TiO₂, while “black silicone” means silicone with an addition of soot particles.

### Example 1

### TOPAS® (ethylene-norbornene copolymers)

22 g of TOPAS 6017S-04 was dissolved in 275 g of chloroform. Next, 0.5 g of Pt(II) meso-tetra(pentafluorophenyl)porphine (CAS-No.: 109781-47-7) was added to the solution. After a short heating and cooling, the solution was ready for film formation. Pt(II) meso-tetra(pentafluorophenyl)porphine is particularly well suited for measuring high concentrations of oxygen.

As an alternative, the analogous Pd(II) compound was used, i.e. Pd(II)-meso-tetra(pentafluorophenyl)porphine, which is particularly well suited for measuring low concentrations of oxygen.

As a further alternative, TOPAS was dissolved in cyclohexane and Pd(II)-meso-tetraphenyl tetrabenzoporphine was added.

Alternatively, any of the conventional methods for the film formation from solvents can be used. In a first method, the films were obtained by means of spin coating.

A glass wafer of 5×5 cm² and 1 mm thickness was cleaned thoroughly and then pretreated with an alcoholic solution of glycidyloxypropyl-trimethoxysilane to achieve a better adhesion of the polymer matrix.

Next, the spin coater (Lot Oriel, Model SCI-20) was loaded with the wafers. To achieve a more uniform film formation, the wafer surface had to be completely covered with fluorophor-polymer solution. The solution which covered the entire wafer was then spun off with a rate of rotation of 3000 rpm which was reached with an acceleration of 2000 rpm/sec. In the process a relatively large amount of the expensive platinum-fluorophor-containing polymer solution is spun off and used up. In order to nevertheless achieve an economical film formation process, the spun-off solution was preferably recaptured, dissolved again, and thus recycled.

With this method, it was possible to obtain films with a thickness of about 5 μm±0.2 μm.

In order to counteract the tendency of the solvent towards film formation, it was also attempted to form the films in a solvent-saturated atmosphere inside a chamber. With this approach solutions could be spun out from the center of the wafer by dispensing 3 ml of the fluorophor-polymer solution. This resulted in a coating thickness of about 5.3 μm, albeit with a somewhat larger variation in film thickness.

As an alternative, the wafers were coated with the fluorophor-polymer solution in a dip-coating or immersion bath process. The wafers were immersed in the fluorophor-polymer solution and subsequently hung up to allow the membrane layer to dry. A problem that occurred with this method was that the solution would accumulate about 2 to 3 mm from the lower edge of the wafer, leading to an increased film thickness in this area. These parts of the wafers would be rejected later in the process because of the layer thickness being too large and/or too uneven. This method produced coating layers of somewhat less uniform thickness around 6 μm, with the layer thickness slightly increasing towards the bottom edge of the wafer.

Under a third method, the fluorophor-polymer solution is sprayed by means of a spray coating process over the wafer. The wafers are sprayed from some distance up to a point below the viscous flow limit of the solution. For somewhat thicker layers and higher viscous flow limits and/or better stability, the fluorophor-polymer solution can also be set up in a slightly higher concentration, which rapidly increases the viscosity of the solution. This method produced the most uniform and thinnest films with thicknesses of e.g. 3.1 μm±0.1 μm.

Under a fourth method, the films were produced by means of a blade-coating machine using so-called thick-film technology. The blade-coating machine was set for a slot width (distance between coating blade and wafer substrate) of 60 μm and films were drawn from the fluorophor-polymer solution. The solvent was subsequently allowed to evaporate. The resultant films were slightly wavy and had a thickness of about 5.1 μm±0.4 μm.

Under a fifth method, polymer spheres with immobilized fluorophor were produced. These were then imbedded in silicone, and the imbedded spheres were drawn out into a film according to the fourth method. The polymer spheres were obtained by precipitation from fluorophor-polymer solution by adding a further solvent in which the polymer spheres are insoluble and with subsequent centrifugation. Suitable as a further solvent are above all polar solvents such as for example water, acetone, ethanol or mixtures thereof.

Alternatively, polymer spheres can be produced through a process of spray-drying.

As a further alternative, it suggests itself to produce fluorophor-free spheres which are treated subsequently with the fluorophor so that the latter penetrates the spheres by diffusion. These methods of producing spheres from polymer materials are known in principle and will therefore not be described in detail.

The film thickness of the polymer matrix is of importance insofar as it affects two important factors in the optical measurement of oxygen content by means of fluorescence quenching. First, the film thickness determines the emission rate or reflection rate and thus the intensity of the fluorescent response, and secondly it is a governing factor for the response time of the measuring device.

For a good response time it is advantageous if the films are as thin as possible, so that the diffusion path that the oxygen has to travel for quenching the fluorescence is as short as possible. The oxygen diffusion rate through the polymer is the second factor which has an influence on the response times of the sensor elements. The diffusion rate should be as high as possible and is specific to the material. For TOPAS grades, the oxygen diffusion rate is around 250 (for TOPAS 5013X14, Tg=136° C.) and 280 cm³×100 μm/(m²×day×bar) (for TOPAS 6013F-04).

On the other hand, it is necessary for the fluorescent response to have a certain minimum intensity even with low concentrations of the analyte, which calls for a minimally required concentration of the fluorophor to be present in the polymer matrix. Also, with a greater film thickness, the fluorophors will wash out less rapidly, for example under autoclaving. Thus, in order to achieve a strong fluorescent response while keeping the film thickness low, the concentration of fluorophors in the polymer matrix and in the polymer solution should on the one hand be high, but still low enough that the polymer properties are not unfavorably affected, in particular Tg, the thermal properties and the diffusion properties through the polymer. Good results were achieved with a fluorophor concentration of 2.3% g/g polymer. Further possible ways to increase the measurement signal through a change of the surface structure of the polymer matrix will be explained in more detail in the context of Examples 4 to 6.

Instead of chloroform, which is highly toxic, one could also use cyclohexane as solvent. The solubility of the TOPAS polymer is even better in this solvent, and it is possible to dissolve the polymer in cold solvent at concentrations of up to 10% g polymer per g. However, the solubility for the fluorophor selected in this example is somewhat lower, so that it is indispensable to heat and then cool the solution prior to film formation. In selecting the solvent, care must be taken in each case that the polymer as well as the fluorophor has sufficient solubility. The solvent should further have a low boiling point, if possible, so that it can also be removed again from the polymer matrix. The solvent needs to be removed carefully in order to prevent bubbles from forming in the polymer matrix. After the films had been made, they were therefore pre-dried sufficiently in air at room temperature prior to drying them in the vacuum-drying cabinet.

After film formation and air-drying during about 24 hours, the coated wafers were dried for one hour at 120° C. in the vacuum-drying cabinet.

The films produced in this manner where then provided alternatingly with white, porous TEFLON® layers (thickness 5 μm) or with white silicone layers as optical coverings or cover layers in order to protect the measuring system from extraneous light and from extraneous fluorescence coming out of the measurement medium. In order to achieve rapid response times and to avoid an increase in film thickness with additional white silicone layers while still producing good reemission values, the sensitive film can also be covered with a white, porous paper or blotting sheet. Additionally in some cases a further black silicone- or TEFLON® cover layer was put on top in order to further reduce the intrusion of unwanted extraneous light into the sensor element.

Subsequently, after determining the Stern-Vollmer characteristic, measurements of oxygen in the gas phase as well as in liquids were performed with sensor elements of this kind, which are also referred to as spots. The fluorophors were excited by means of a green LED and appropriate optical filters at a wavelength of about 505 nm with a sinusoidal excitation signal. From the fluorescent response at 650 nm, a phase shift was subsequently determined.

When using Pt(II) meso-tetra(pentafluorophenyl)porphine as fluorophor, the following results were obtained: The UV/VIS spectrum of the fluorophor Pt(II) meso-tetra(pentafluoro-phenyl)porphine (CAS-No.: 109781-47-7) exhibits a first absorption maximum at about 492 nm and two further, weaker maxima at about 507 nm and about 540 nm.

The spectrum of the immobilized fluorophor hardly changed. Thus, no harmful effects caused by the matrix due to a possible luminescence could be found as a result of immobilizing the fluorophor Pt(II) meso-tetra(pentafluoro-phenyl)porphine.

For the oxygen elements containing Pt(II) meso-tetra(penta-fluorophenyl)porphine which were produced according to the foregoing methods, the following response times were measured before and after autoclaving in the gas phase as well as in solution:

| | | | | | | | Response Time/Photomultiplier Counts | | | |
|---|---|---|---|---|---|---|---|---|---|---|
| | | | | | | | | | after 30 autoclaving cycles | |
| | | | Optical Cover | | | | prior to autoclaving | | 130° C. 30 min. | |
| Method of Film | Film | | Teflon | Silicone | Silicone | Silicone | Response Time | Counts | Response Time | Counts |
| Formation | Thickness | | 5 μm | 10 μm | 50 μm | 100 μm | */** (s) | in O₂ | */** (s) | in O₂ |
| Spin-Coating | 5 | μm | X | | | | 20/45 | 17000 | 22/46 | 15000 |
| Spin-Coating | 5 | μm | | X | | | 31/59 | 19000 | 30/61 | 17500 |
| Spin-Coating | 5 | μm | | | X | | 42/71 | 19000 | 40/73 | 18000 |
| Spin-Coating | 5 | μm | | | | X | 55/92 | 19000 | 55/95 | 18000 |
| Dip-Coating | ca. 6 | μm | X | | | | 25/48 | 18000 | 30/50 | 15500 |
| Dip-Coating | ca. 6 | μm | | X | | | 35/62 | 20000 | 36/64 | 18000 |
| Dip-Coating | ca. 6 | μm | | | X | | 45/80 | 20000 | 50/85 | 18000 |
| Dip-Coating | ca. 6 | μm | | | | X | 60/122 | 20000 | 68/142 | 18500 |
| Spray-Coating | 3.1 | μm | X | | | | 15/38 | 12000 | 20/41 | 11000 |
| Spray-Coating | 3.1 | μm | | X | | | 24/55 | 13500 | 28/60 | 12000 |
| Spray-Coating | 3.1 | μm | | | X | | 38/67 | 13500 | 41/69 | 12500 |
| Spray-Coating | 3.1 | μm | | | | X | 50/88 | 13500 | 51/86 | 12500 |
| Blade-Coating | 5.1 | μm | X | | | | 22/46 | 17000 | 22/49 | 15000 |
| Blade-Coating | 5.1 | μm | | X | | | 31/61 | 19000 | 34/66 | 17000 |
| Blade-Coating | 5.1 | μm | | | X | | 45/75 | 19000 | 50/83 | 17500 |
| Blade-Coating | 5.1 | μm | | | | X | 59/104 | 19000 | 101/114 | 18000 |

*/** Response times t_{98%} were determined a) by changing the medium from air to nitrogen and from nitrogen to air, and b) by changing the medium from oxygen-saturated water to oxygen-free water (water + sodium disulfite + trace of cobalt).

The detection limit for the fluorescence yield with the measuring instrument that was being used was about 5000. If the count falls below this value, the analyte concentration is below the detection limit and/or the sensor element needs to be replaced.

The response times grew longer by only an insignificant amount after 30 autoclaving cycles. The thicker the films and also in particular the silicone layers, the longer the response times became. Measurement spots could be produced with good response times that are fast enough for sterilization processes in the food- and pharmaceuticals industries even after several autoclaving cycles. Also, the intensity of the measured fluorescence of the spots was hardly decreased by the autoclaving process (in phosphate buffer), the fluorophors were not washed out to any significant extent and hardly bleached out by the thermal treatment. It is known that the dye by itself is temperature-resistant. Its strong resistance against washing-out and degradation of the fluorophor is attributed to the high glass transition temperature of the immobilizing polymer and also to its low degree of water absorption, especially at high temperature levels.

### Example 2

### Optochemical Sensor Element with Polymer Wafers

A further example of an optochemical sensor element concerns the use of polymer wafers as substrates. Polymer wafers as substrates improve the adhesion on the substrate for the polymer matrix with the immobilized fluorophor, particularly after aging or after a large number of autoclaving cycles. Furthermore, the wetting of the wafer during the spin-coating process and thus the uniformity of the film is critically influenced by the matching of the hydrophilic properties between the substrate and the spun-out solvent with the dissolved polymer and fluorophor.

Polymeric wafers are considerably more hydrophobic than glass surfaces, and the match between the respective hydrophilic properties of the solvent, in this case chloroform or cyclohexane, and the wafer substrate is significantly better, which leads to better wetting of the wafer and thus more homogeneous and topographically uniform film surfaces. Also, the adhesion of the spun-out polymer films is better on the polymeric wafers than on silanized glass surfaces. This makes it absolutely unnecessary to assemble a layered structure by bonding the individual layers together with an adhesive.

The improved adhesion is on the one hand due to an improved match between the respective hydrophilic properties of the polymer film or polymer matrix and the underlying wafer substrate and on the other hand due to the fact that the solvents in part attack these wafers superficially, causing the topmost polymer layer of the wafer to become partially dissolved. As the solvent evaporates, the partially dissolved polymer chains of the wafer will become interlaced with the polymer chains of the deposited film that are still in solution. This leads to an enormously increased adhesive strength and strongly improves the ability of the sensor elements to withstand autoclaving. The following procedure yielded sensor spots of optimal properties:

### Polymer Solution:

An amount of 22 g of TOPAS 6015S-04 was dissolved under reflux in 178 g cyclohexane. After cooling, an amount of 0.46 g of Pd(II)-meso-tetra(pentafluorophenyl)porphine was added to the solution. In order to better dissolve the fluorophor, the solution had to be brought to boil again for a short time. Next, the film formation took place by means of the spin-coating process on polymer wafers such as semi-crystalline PA (for example EMS Chemicals, type TR XE 3942, Tg=190° C.; maximum service temperature 160° C., autoclavable at 130° C.; wafer thickness 0.8 mm), or TOPAS itself (Type 6015S-04; thickness 0.8 mm-1 mm; maximum service temperature >145° C.).

This led to the following observations:

The spun-out solution was spreading better and more evenly over the surface and produced more uniform films than were obtained with spin-coating of untreated glass wafers or silanized glass wafers.

Due to the better match between the respective hydrophilic properties of the solvent and the wafer substrate the wetting of the wafer is significantly better and faster, so that the films forming on the surface are free of strong topographical variations. Preferably, the TOPAS wafers were spun off directly after applying the solution under a solvent-saturated atmosphere. Topas is superficially etched by contact with the cyclohexane, which improves the adhesion of the spun-out film. It was possible in this way to build up clear transparent spots.

Polyamide wafers (PA wafers) are significantly more stable against cyclohexane and can at most be superficially etched by the latter. Among the polymer materials, COC/TOPAS in particular has good optical transparency in the lower wavelength range, which is important in particular for the short excitation wavelengths of the fluorophors (up to 400 nm). As a result, the fluorescence yield is increased as there is less absorption of the fluorescence in the carrier material of the optochemical sensor element.

After drying in air and oven-drying at 120° C., the wafers were subjected to an initial autoclaving cycle.

After application of an optical cover layer of white silicone of about 60 μm thickness, analogous measurements as in Example 1 were made on the sensor spots. The resultant response times were found to be about 30 seconds in the gas phase and about 80 seconds in condensed phases.

With a count of about 14000 (in air) for the sensor spots on TOPAS wafers, the fluorescent yield was high enough, while the spots on PA wafers had counts of only about 9000, which is due to the lower transparency of the PA wafers.

The spots were next subjected to aging processes and especially to repeated CIP cycles, wherein the cleaning was performed with nitric acid as well as with caustic soda (5% NaOH for 70 hours at 80° C. or 5% HNO₃ for 70 hours at 80° C.). This demonstrated the outstanding stability of the polymer COC against bases and acids. The PA wafers exhibited no change after the treatment with caustic soda, but they yellowed in nitric acid. The PA wafers that had turned yellow after the acid treatment showed an increased absorption rate which caused a deterioration of the sensor element characteristic. In contrast, the COC wafers showed no loss of transmissivity and no discoloration after CIP procedures, regardless of whether they were performed in a base or an acid.

### Example 3

### Optochemical Sensor Element with Hybrid Wafers

In a further example, hybrid wafers are used as an alternative to the polymer wafers of Example 2. Hybrid wafers in essence comprise of an oxygen-impermeable glass which comprises a polymeric adhesion agent for providing a good adhesion of the fluorophor-polymer solution that is to be applied by spin-coating. The adhesion agent was preferably spread very thin, allowing it to sufficiently interact with the solvent during spinning out a polymer film, while the oxygen reservoir resulting from the adhesion agent is kept as low as possible. Adhesion agents should not exceed a layer thickness of 10-20 μm, have a very low oxygen solubility, and they should also be transparent. In this way, a cross-linking becomes possible between the polymer chains of the superficially etched adhesion agent and the polymer that is to be spun out, resulting in sensor elements that keep their good adhesion even after aging, which can be produced in an analogous way as the sensor elements described in example 2.

An advantage in using hybrid wafers is that, in contrast to polymer wafers, they exhibit essentially no oxygen permeability and in addition, drift-free sensor elements can be realized. In addition, with the low oxygen reservoir, the response time of the sensor element is shortened. First, a TOPAS layer or an epoxy layer was applied as adhesion agent.

TOPAS as adhesion agent was applied by spin-coating as a layer of approximately 10 μm thickness to a hybrid wafer. Having been pretreated in this manner, the wafer was subsequently processed as described in Example 1.

Epoxy as adhesion agent is in essence optically transparent, and epoxides show in addition especially low oxygen solubility, so that thicker layers can also be realized. As adhesion agents, one could use for example the low-viscosity epoxy products which are distributed by EPOTEK under the trade names EPOTEK 301, 302, 302FL and can be spun out into thin layers onto glass wafers by means of a spin coater.

The following is a description of how the adhesion agent layer of epoxy was put onto a glass wafer which was subsequently processed further according to the method of Example 1:

To achieve a better wetting of the epoxy layers, the glass wafer was first pretreated with epoxy propyl trimethoxysilane. In this pretreatment, the silane group reacts on the one hand with the glass surface, and on the other hand a covalent bond is established between the epoxy group of the silane and the epoxy adhesion agent. On said pretreated wafer smooth epoxy films can be spun out.

Next, EPOTEK 301 was spun out on the pretreated glass wafer with a final speed of about 3000 rpm. In order to achieve optimal adhesion of the subsequently applied fluorophor-polymer layer, the spun-out epoxy was first only partially hardened for one hour at about 50° C., in this case to about two thirds of its final hardness, and fully hardened only together with the fluorophor-polymer layer after the latter had been applied. The time for hardening EPOTEK 301 is around one hour at 65° C., or 24 hours at 23° C.

### Example 4

### Sensor Film Applied to Optical Fiber

In a further example, the polymer matrix with the immobilized fluorophor was applied as cladding or part of the cladding to the core of a POF optical fiber. An optical fiber normally consists of a light-conducting core which is surrounded by a cladding and a jacket. The jacket serves primarily as outward protection of the optical fiber.

It should be noted that the polymer in which the fluorophor is immobilized needs to have a smaller refractive index than the material of the underlying core, so that total internal reflection will occur at the interface between core and cladding. The fluorophor in the polymer matrix is in this case excited by the evanescent field of the optical fiber. The core should be a material with a refractive index of about 1.6 to 1.8. Suitable optical fibers are for example those with a core of poly(pentabromophenyl acrylate-co-glycidyl methacrylate) (glass transition temperature of 162° C., soluble in chloroform, Fluka Article No. 591408), which has a high refractive index.

This means that as polymer matrix with immobilized fluorophor or polymer cladding all polymers can be used which have a refractive index of less than 1.6 and which, in addition, have the required thermal and optical properties.

These include COC, COP as well as PMMI polymers which have the following refractive indices (ISO 489):

COC (Type 60155S-04) n=1.53
PMMI (PLEXIMID 8817) n=1.53

### Procedure:

The jacket and the cladding were removed from the POF optical fiber over a length of about 3 mm. Then the underlying core material of poly(pentabromophenyl acrylate-co-glycidyl methacrylate) was cleaned with ethanol.

Next, the tip was coated by dip-coating in a fluorophor-polymer solution of very low viscosity (6.25% Topas dissolved in chloroform, 2.5% fluorophor g/g polymer). The polymer drop at the end of the glass fiber which remained after the dip coating was cut off with a knife and the now exposed fiber end was coated with silver as a reflector. Then the entire fiber ending (tip with silver coating and the 3 mm of polymer coating with immobilized fluorophor) was covered with an oxygen-permeable covering or jacket.

The covering should again have a smaller refractive index than the underlying polymer cladding. The covering should furthermore be oxygen-permeable and elastic in order to offer good protection for the core and the cladding. An example of an oxygen-permeable covering of this kind is white RTV silicone (silicone of vinyl-terminated poly(dimethylsiloxane)) whose use as optical cover was already described in the preceding examples 1 to 3. The silicone in this case is preferably white in order to prevent the unwanted intrusion of extraneous light into the optical fiber. The covering was applied with a layer thickness of at most about 100 μm in order to realize sensor elements with a short response time.

Products that can be considered for this kind of covering include silicone products distributed by Gelest, Inc. (Gelest OE™ 41/42/43) with refractive indices of 1.41 to 1.43, filled with titanium dioxide, or a regular low-viscosity RTV silicone from Dow Corning, for example RTV 732 “white”. The latter silicone is available as white silicone and is FDA-approved for contact with food products. Further suitable silicone products are commercially available.

### Applied Polymer Solution:

1 g of TOPAS (type 6015S-04) was dissolved in 15 g of chloroform. Then, 0.0253 g of Pt(II) meso-tetra(pentafluorophenyl)porphine was added to the chloroform solution and thoroughly dissolved. For this purpose, the solution was briefly heated under reflux. Next, the solution was used for the dipping of the pretreated glass optical fibers.

As an alternative, the analogous Pd(II) compound was used, i.e. Pd(II)-meso-tetra(pentafluorophenyl)porphine.

As a further alternative, TOPAS was dissolved in cyclohexane and Pd(II)-meso-tetra(pentafluorophenyl)porphine was added to the solution.

### Result:

Due to dipping in a relatively viscous silicone solution, the optical protection layers, specifically the covering, for the optical fibers turned out relatively thick, and this had an effect on the response times of the sensor elements made with this method. Due to its lower viscosity, the Gelest product resulted in thinner coverings and thus faster responding sensor elements than were obtained by coating the optical fibers with Silicone (Type 732) “white”. On the other hand, the optical fiber with the Gelest coating is more sensitive to extraneous light, with the compound being applied in the form as received from Gelest, i.e. free of TiO₂ and thus transparent. For further experiments, a white TiO₂ layer should be introduced as a light barrier. Due to the transparency of the material, the measurements had to be performed in the absence of extraneous light. With both kinds of optical covering, sensor elements with an adequate fluorescence signal could be obtained.

Due to the chloroform solubility of the core material poly(pentabromophenyl acrylate-co-glycidyl methacrylate) and its thermal stability, it was possible to achieve good adhesion between the core material and the applied polymer cladding. It could be demonstrated that optical fibers prepared in this way as sensor elements are able to withstand autoclaving cycles and—if adhesion between the covering is given—can also be used for high-temperature applications up to 130° C. The adhesion between covering and cladding can be achieved or increased analogously as in Example 1.

### Example 5

### Wafer with Structured Surface

In a further example a glass wafer with a structured surface is used as substrate, with either a regular or irregular kind of structuring. The structuring of the wafer can be achieved through sandblasting, grinding or different known etching techniques. By etching with photolithographic methods, it is also possible to produce a regular, defined structure and/or diffusely scattering geometries.

### Procedure:

Structured wafer surfaces with depressions of 50 to 100 μm depth were produced according to one of the known methods.

Next, a fluorophor-polymer solution was spun out on the structured surface of the wafers as in the preceding examples, or alternatively the structured surface was covered with the solution.

After drying of the sensor element which had been produced in this way its surface was ground down so that depressions of about 5 to 20 μm were left in place. As a result of the grinding, free glass areas were created on the structured surface as well as areas that were covered with the polymer matrix containing the immobilized fluorophor. In comparison to the coated areas, the free glass areas had an increased resistance to aging.

As a next step, a radiation impermeable cover layer with a thickness around 50 μm can easily be spread over the surface by blade-coating.

At the differently shaped depressions, the excitation light is scattered diffusely, whereby the measurement signal, in this case the fluorescence intensity fluorescence, is increased.

### Example 6

### Polymer Spheres Immobilized in Silicone

In this last example, spheres of TOPAS with immobilized fluorophor are imbedded in silicone and the resultant polymer mixture is spread onto glass wafers by blade-coating.

### Procedure:

Polymer spheres of the fluorophor-polymer mixtures that have already been mentioned herein are obtained on the one hand by precipitation from the fluorophor-polymer solution dissolved in chloroform or cyclohexane by adding a second solvent, for example water, in which the polymer spheres are insoluble, and subsequent centrifugation. The spheres obtained by this process were subsequently imbedded in a silicone film.

Alternatively, fluorophor-polymer films with a thickness of less than 10 μm were spun out non-adhesively on glass substrates. The resultant films were dissolved or peeled off from the substrates and broken up into small pieces. The fragments of the polymer matrix were then introduced into silicone films.

### Result:

It could be demonstrated that the size or the size distribution of the polymer spheres has an influence on the response time of the resultant sensor element. The smaller the spheres, the shorter or faster were the response times of the resultant sensor elements. The thickness of the silicone layers did not have a big influence on the response time. It was shown that sufficiently short response times could be realized even with a total layer thickness of the sensor element of about 100 μm.

What is claimed is:

1. A sensor element for optochemically measuring gaseous or dissolved analytes, particularly oxygen, the sensor element comprising:
a polymer matrix comprising a polymer that has a non-aromatic backbone and that is selected from the group consisting of: ethylene-norbornene copolymer, cyclic olefin polymers, and poly(n-methyl methacrylimide); and
a fluorophor, immobilized in the polymer matrix.

2. The optochemical sensor element of claim 1, wherein:
the polymer is selected from the group consisting of poly(n-methylmethacrylimide) and ethylene-norbornene copolymer.

3. The optochemical sensor element of claim 2, wherein:
the polymer is an ethylene-norbornene copolymer.

4. The optochemical sensor element of claim 1, further comprising:
a substrate, on which the polymer matrix is arranged.

5. The optochemical sensor element of claim 4, wherein:
the substrate comprises a material selected from the group consisting of: glass, polyester, amorphous or partially crystalline polyamide, acrylate, polycarbonate, cyclic olefin copolymers and combinations of these materials.

6. The optochemical sensor element of claim 5, wherein:
the substrate comprises the cyclic olefin copolymer ethylene-norbornene copolymer.

7. The optochemical sensor element of claim 5, wherein:
the substrate is a hybrid substrate.

8. The optochemical sensor of claim 1, further comprising:
an optical fiber, with the polymer matrix arranged directly on a core of the fiber.

9. The optochemical sensor element of claim 1, further comprising:
a silicone matrix, with spheres or fragments of the polymer matrix arranged therein.

10. A sensor element for optochemically measuring gaseous or dissolved analytes, particularly oxygen, the sensor element comprising:
a polymer matrix comprising ethylene-norbornene copolymer; and
a fluorophor, immobilized in the polymer matrix.

11. The optochemical sensor element of claim 10, further comprising:
a substrate, on which the polymer matrix is arranged.

12. The optochemical sensor element of claim 11, wherein:
the substrate comprises a material selected from the group consisting of: glass, polyester, amorphous or partially crystalline polyamide, acrylate, polycarbonate, cyclic olefin copolymers and combinations of these materials.

13. The optochemical sensor element of claim 12, wherein:
the substrate comprises the cyclic olefin copolymer ethylene-norbornene copolymer.

14. The optochemical sensor element of claim 12, wherein:
the substrate is a hybrid substrate.

15. The optochemical sensor of claim 10, further comprising:
an optical fiber, a core of which has the polymer matrix arranged directly thereon.

16. The optochemical sensor element of claim 10, further comprising:
a silicone matrix, with spheres or fragments of the polymer matrix arranged therein.

17. A method for determining concentration of a gaseous or dissolved analyte in a medium, comprising the steps of:
providing a measuring device comprising an optochemical sensor element of claim 1;
operatively inserting the measuring device into the medium; and
determining the concentration of the gaseous or dissolved analyte present in the medium, using the measuring device.

18. The method of claim 17, wherein:
the determined analyte is oxygen.
